(19) United States
(12) Patent Application Publication
(10) Pub. No.: US 2023/0129137 A1
(43) Pub. Date: Apr. 27, 2023

(54) USE OF TT-10-LOADED NANOPARTICLES FOR CARDIAC REPAIR

(71) Applicant: THE UAB RESEARCH FOUNDATION, Birmingham, AL (US)

(72) Inventors: Jianyi Zhang, Vestavia Hills, AL (US); Yang Zhou, Vestavia Hills, AL (US)

(21) Appl. No.: 17/968,113

(22) Filed: Oct. 18, 2022

Related U.S. Application Data
(60) Provisional application No. 63/270,721, filed on Oct. 22, 2021.

(51) Int. Cl. A61K 9/51; A61K 31/427; A61P 9/10

(52) U.S. Cl. CPC A61K 9/5153; A61K 31/427; A61P 9/10

(57) ABSTRACT

In one aspect, the disclosure relates to nanoparticle compositions including TT-10 or a pharmaceutically acceptable salt thereof and nanoparticles including at least one biocompatible polymer or copolymer such as poly-lactic-co-glycolic acid. Also disclosed are methods for improving at least one cardiac function or property following heart damage in a subject, the methods including at least the step of administering the nanoparticle compositions to the subject. In another aspect, the nanoparticle compositions can be administered by intraperitoneal injection and are active at a target site for a period of at least 4 weeks.

### CROSS REFERENCE TO RELATED APPLICATIONS

This application claims the benefit of U.S. Provisional Application No. 63/270,721, filed on Oct. 22, 2021, which is incorporated herein by reference in its entirety.

### BACKGROUND

The endogenous regenerative capacity of adult mammalian hearts is exceptionally limited and cannot replace cardiomyocytes that are lost to myocardial infarction (MI) or other cardiac disorders. However, when apical resection surgery was performed in mice or pigs shortly after birth, and MI was experimentally induced several weeks later, the animals recovered completely with little evidence of scarring. Furthermore, the recovery appeared to be driven primarily by the proliferation of pre-existing cardiomyocytes, suggesting that postnatal cardiomyocytes retain some latent capacity for proliferation, and that strategies targeting mechanisms of cardiomyocyte cell-cycle regulation could lead to the development of promising new therapies for heart disease.

Hippo signaling has a key regulatory role in fetal cardiac development, and two downstream components of the Hippo pathway, YES-associated protein (YAP) and transcriptional coactivator with PDZ-binding motif (TAZ), have recently been linked to cardiac regeneration. The pharmaceutical product TT-10 (C₁₁H₁₀FN₃OS₂) is a fluorine substituent of TAZ-12 and appears to promote cardiomyocyte proliferation: in mice, intraperitoneal injections of TT-10 reduced infarct size one week after MI but did not prevent progressive declines in cardiac function at later time points. Although fluorine-containing pharmaceuticals such as TT-10 are metabolically stable and have high bioavailability, many such compounds have poor retention at the site of administration. Ideally, a therapy for MI would allow for continued release of active agents in such as TT-10 over time without the need for repeated intraperitoneal injections.

Despite advances in initial treatment of cardiac damage caused by MI and other injuries to cardiac tissue, there is still a scarcity of compositions and methods for treating progressive declines in cardiac function following MI and other cardiac events. An ideal method would involve release of active agents at a target location over time in order to promote cardiomyocyte proliferation and maintain high levels of cardiac function in MI and heart disease patients while avoiding systemic side effects associated with higher doses of the active agents. These needs and other needs are satisfied by the present disclosure.

### SUMMARY

In accordance with the purpose(s) of the present disclosure, as embodied and broadly described herein, the disclosure, in one aspect, relates to nanoparticle compositions including TT-10 or a pharmaceutically acceptable salt thereof and nanoparticles including at least one biocompatible polymer or copolymer such as poly-lactic-co-glycolic acid. Also disclosed are methods for improving at least one cardiac function or property following heart damage in a subject, the methods including at least the step of administering the nanoparticle compositions to the subject. In another aspect, the nanoparticle compositions can be administered by intraperitoneal injection and are active at a target site for a period of at least 4 weeks.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1E show TT-10 increased cell cycle activity and proliferation in cultured human pluripotent stem sell derived cardiomyocytes (hiPSC-CMs). (FIG. 1A) TT-10 (C₁₁H₁₀FN₃OS₂; molecular weight, 283.34 Da) contains 4 C═C, 1 C═N, and 1 C═O double bond. Maximum UV/Vis absorbance is approximately 200 nm. (FIGS. 1B-1E) hiPSC-CMs were cultured under standard conditions (—) or treated with varying concentrations of TT-10 (2 μM, 10 μM, 20 μM, or 100 μM) for 48 hours and immunofluorescently stained for the human isoform of cardiac troponin T (hcTnT). Nuclei were identified by staining with 4′,6′-diamidinio-2-phenylindole (DAPI) or with DAPI and Nkx2.5 antibodies. (FIG. 1B) Proliferation was evaluated via immunofluorescence costaining for antigen KI 67 (Ki67) and quantified as the percentage of Ki67-positive cells. (FIG. 1C) hiPSC-CMs in the S phase of the cell cycle were identified via immunofluorescence costaining for bromodeoxyuridine (BrdU) incorporation and quantified as the percentage of BrdU-positive cells. (FIG. 1D) hiPSC-CMs in the M phase of the cell cycle were identified via immunofluorescence costaining for histone 3 phosphorylation (PH3) and quantified as the percentage of PH3-positive cells. (FIG. 1E) hiPSC-CMs undergoing cytokinesis were identified via immunofluorescence costaining for Aurora B (AuB) and quantified as the percentage of AuB-positive cells. Scale bar: 20 μm. All experiments were repeated 5 times. *P<0.01, **P<0.05, 1-way ANOVA with Tukey's correction.

FIGS. 2A-2B show TT-10 enhanced survival and nuclear Yap levels in cultured hiPSC-CMs. (FIG. 2A) Apoptosis was evaluated via TUNEL staining and quantified as the percentage of TUNEL-positive cells. (FIG. 2B) hiPSC-CMs were immunofluorescently costained for Yap, and the proportion of cells with Yap-positive nuclei is presented as a percentage. Scale bar: 20 μm. All experiments were repeated 5 times. *P<0.01, Student's t test.

FIGS. 3A-3D show cultured TT-10 nanoparticles (TT-10-NPs) released TT-10 for more than 4 weeks and were taken up by hiPSC-CMs. (FIG. 3A) poly(lactic-co-glycolic acid) PLGA NPs were imaged via scanning electron microscopy. A representative image of a TT-10-NP is shown. Scale bar: 200 nm. (FIG. 3B) The size distribution of the empty PLGA NPs (Empty-NP) (left) and TT-10-NPs (right) was evaluated via nanoparticle tracking analysis. (FIG. 3C) The kinetics of TT-10 release from TT-10-NPs were determined with a dialysis device. Suspensions of TT-10-NPs were incubated at 37° C., and samples of the elution medium were withdrawn and replaced at the indicated time points. Then, the cumulative percentage of TT-10 that had been released from the NPs was calculated for each time point. (FIG. 3D) hiPSC-CMs were cultured with NPs (2 μg/mL) that had been loaded with coumarin-6 (green) for 24 hours or 4 weeks. Then, the cells were immunofluorescently stained for human cardiac troponin T (hcTnT) (red), and nuclei were identified by DAPI staining (blue). Internalized NPs are identified with arrows. Scale bar: 20 μm.

FIGS. 4A-4H show Intramyocardial injections of TT-10-NPs improved recovery from MI in mice. MI was induced in mice, and then the animals were treated with Dulbecco's phosphate buffered saline (DPBS), empty PLGA NPs (Empty-NP), TT-10 solution (TT-10-SOL), or with TT-10-loaded PLGA NPs (TT-10-NP). A fifth group of animals (the sham group) underwent all surgical procedures for MI induction except arterial ligation. (FIG. 4A) Echocardiographic assessments of (FIG. 4B) left ventricular ejection fraction (LVEF), (FIG. 4C) fractional shortening (LVFS), (FIG. 4D) end-systolic diameter (LVESD), and (FIG. 4E) end-diastolic diameter (LVEDD) were conducted before MI induction (pre-s) and 1 and 4 weeks afterward. Representative images in A were collected at week 4. (FIGS. 4F-4H) Animals were sacrificed at week 4, and hearts were explanted. (FIG. 4F) Heart sections were stained with Picrosirius Red and Fast Green to identify regions of infarcted (red) and noninfarcted (green) tissue. Scale bar: 1 mm. (FIG. 4G) Then, infarct sizes were quantified as the ratio of the scar area to the total left ventricular surface area and expressed as a percentage. (FIG. 4H) Myocardial hypertrophy was evaluated as the ratio of the weight of the whole heart to the animal's bodyweight (HW/BW). (FIGS. 4B-4H) n=7-9 animals per group. *P<0.01 vs. DPBS, ^{†}P<0.05 vs. DPBS, ^{#}P<0.01 vs. Empty-NP, ^{&}P<0.01 vs. TT-10-SOL; 2-way ANOVA with Tukey's multiple comparisons test. (G and H) n=7-9 animals per group; ^{¥}P<0.01 vs. sham, ^{†}P<0.05 vs. sham, *P<0.01 vs. MI, ^{#}P<0.01 vs. Empty-NP, ^{&} P<0.01 vs. TT-10-SOL; 1-way ANOVA with Tukey's multiple comparisons test.

FIGS. 5A-5D show intramyocardial injections of TT-10-NPs after MI increased cell cycle activity, proliferation, nuclear Yap abundance, and survival in cardiomyocytes. Sections from the border zones of the infarcts in animals from the DPBS, Empty-NP, TT-10-SOL, and TT-10-NP groups, and from the corresponding region of hearts from sham animals, were obtained (FIGS. 5A-5C) 1 week or (FIG. 5D) 72 hours after MI induction and treatment or sham surgery. Then, the sections were stained for cardiac troponin T (cTnT) to visualize cardiomyocytes, and nuclei were identified via DAPI staining. Scale bar: 20 μm. n=5 animals per group, 4 sections per heart, and 5 high-power fields per section. (FIG. 5A) Cardiomyocyte proliferation was evaluated via immunofluorescence costaining for Ki67 and quantified as the percentage of cTnT-positive cells that also expressed Ki67. (FIG. 5B) Cardiomyocytes in the M phase of the cell cycle were identified via immunofluorescence costaining for PH3 and quantified as the percentage of cTnT-positive cells that were also positive for PH3. Representative images in FIGS. 5A-5B were obtained at week 1. (FIG. 5C) Sections were immunofluorescently costained for Yap, and the proportion of cTnT-positive cells with Yap-positive nuclei is presented as a percentage. (FIG. 5D) Cardiomyocyte apoptosis was evaluated via TUNEL staining and quantified as the percentage of cTnT-positive cells that were also positive for TUNEL. ^{¥}P<0.01 vs. sham, ^{†}P<0.05 vs. sham, *P<0.01 vs. DPBS, ^{#}P<0.01 vs. Empty-NP, ^{&}P<0.01 vs. TT-10-SOL; 1-way ANOVA with Tukey's multiple comparisons test.

FIG. 6 shows Assessment of TT-10-NP-mediated angiogenesis in the border zone of the infarct. Sections from the border zone of animals that underwent MI induction, or from the corresponding regions of hearts from sham-operated animals, were obtained at week 4 and stained with the endothelial marker isolectin B4 (IB4) and for the expression of α smooth-muscle actin (α-SMA). Nuclei were identified via DAPI staining, and then vascular density and arteriole density were quantified by determining the number of IB4-positive and α-SMA-positive vascular structures, respectively, per square millimeter. Scale bar: 50 μm. n=7-9 animals per group, 4 sections per heart, and 5 high-power fields per section. ^{¥}P<0.01 vs. sham, *P<0.01 vs. DPBS, ^{#}P<0.01 vs. Empty-NP, ^{&}P<0.01 vs. TT-10-SOL; 1-way ANOVA with Tukey's multiple comparisons test.

FIG. 7 shows TT-10 activated the YAP signaling pathway in cultured hiPSC-CMs. hiPSC-CMs were treated with 10 μM TT-10 for 48 hours, and then YAP and phosphorylated YAP (p-YAP) protein abundance was evaluated via Western blot and quantified via normalization to the abundance of glyceraldehyde phosphate dehydrogenase (GAPDH). *P<0.05, Student's t-test; n=3 experiments.

FIGS. 8A-8B show TT-10-NPs promoted proliferation in cultured hiPSC-CMs. hiPSC-CMs were cultured for 48 hours with or without 1 mg/mL coumarin-6-loaded TT-10-NPs (green); then, the cells were immunofluorescently stained for the human isoform of cardiac troponin I (hcTnI, gray), and nuclei were identified via DAPI staining (blue). Internalized NPs are identified with an arrow. Scale bar: 20 μm. (FIG. 8A) Proliferation was evaluated via immunofluorescence costaining for Ki67 and quantified as the percentage of Ki67-positive cells. (FIG. 8B) hiPSC-CMs in the M-phase of the cell cycle were identified via immunofluorescence costaining for PH3 and quantified as the percentage of PH3-positive cells. *P<0.01, Student's t-test; n=5 experiments.

FIG. 9 shows TT-10-NPs remain stable after administration to infarcted mouse hearts. NPs were loaded with both TT-10 and coumarin 6 and then intramyocardially injected into infarcted mouse hearts. Animals were sacrificed 1, 3, 7, 14, or 28 days later; then, sections from the BZ were stained with DAPI, and images of coumarin 6 fluorescence were collected. Scale bar: 200 μm (n=2 per time point).

FIGS. 10A-10K show intramyocardial injections of TT-10-NP improved cardiac function and reduced infarct size in infarcted pig hearts. Acute myocardial infarction was induced by ligating left anterior descending (LAD) coronary artery of Yorkshire pigs (˜15 kg body weight), following by injecting of TT-10-NP (0.5 mg/kg body weight) or equal amount of delivery vehicle (1 mL PBS). FIG. 10A: Echocardiographic images were obtained for pigs in the Vehicle and TT-10-NP groups before surgery, on Day 7 and Day 28 after AMI induction and used to calculate (FIG. 10B) LVEF and (FIG. 10C) LVFS. FIGS. 10D-10H: cMRI was performed on Day 28 and used to calculate (FIG. 10E) LVEF, (FIG. 10F) Stroke Volume, (FIG. 10G) End-systolic Volume, and (FIG. 10H) End-diastolic Volume. FIGS. 10I-10J: LGE-cMRI images were obtained in pigs on Day 28 after MI induction and used to calculate (FIG. 10J) infarct size and (FIG. 10K) infarct mass.

FIGS. 11A-11B show intramyocardial injections of TT-10-NP promoted cardiomyocyte cell-cycle activity in a pig AMI model. Sections of heart tissue from the border zone and remote zone of the infarct were collected from pigs 28 days after MI and stained for the presence of cTnT and (FIG. 11A) Ki67 or (FIG. 11B) PH3; then, cardiomyocyte proliferation and cell-cycle activity were quantified as the percentages of cTnT-positive cells that were also positive for Ki67 and PH3, respectively. Quantified data of Ki67 and PH3 were obtained from 4 sections per cardiac ring, 5 high-power viewing fields per section; 2 border zone cardiac rings and 1 remote cardiac ring were examined. Bar=50 μm. *P<0.05, ***P<0.001.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are nanoparticle compositions including TT-10 or a pharmaceutically acceptable salt thereof and nanoparticles including at least one biocompatible polymer or copolymer such as, for example, poly-lactic-co-glycolic acid (PLGA). In one aspect, the PLGA can have a molecular weight of from about 7000 Da to about 120,000 Da, or of about 7000; 10,000; 15,000; 20,000; 25,000; 30,000; 35,000; 40,000; 45,000; 50,000; 55,000; 60,000; 65,000; 70,000; 75,000; 80,000; 85,000; 90,000; 95,000; 100,000; 105,000; 110,000; 115,000; or about 120,000 Da, or a combination of any of the foregoing values, or a range encompassing any of the foregoing values, where any of the foregoing values can be an upper or a lower endpoint of the range. In some aspects, the PLGA can have a ratio of lactide to glycolide of from about 3:1 to about 1:1, or of about 3:1, 2.5:1, 2:1, 1.5:1, or about 1:1, or a combination of any of the foregoing values, or a range encompassing any of the foregoing values, where any of the foregoing values can be an upper or a lower endpoint of the range.

In some aspects, the nanoparticles encapsulate the TT-10. In one aspect, the nanoparticles can have an average diameter of from about 75 nm to about 250 nm, or of about 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or about 250 nm, or a combination of any of the foregoing values, or a range encompassing any of the foregoing values, where any of the foregoing values can be an upper or a lower endpoint of the range. In one aspect, the nanoparticle composition includes from about 2 μM to about 100 μM of TT-10 or the pharmaceutically acceptable salt thereof, or about 2, 5, 10, 15, 20, 25, 30, 35, 40, 45, 50, 55, 60, 65, 70, 75, 80, 85, 90, 95, or about 100 μM, or a combination of any of the foregoing values, or a range encompassing any of the foregoing values, where any of the foregoing values can be an upper or a lower endpoint of the range.

Also disclosed are methods for improving at least one cardiac function or property following heart damage in a subject, the methods including the step of administering the nanoparticle compositions to the subject. In one aspect, the subject is a human. In another aspect, the nanoparticle compositions can be administered by intraperitoneal injection, oral administration, parenteral injection, intramuscular injection, intravenous administration, or any combination thereof, and are active at a target site for a period of at least 4 weeks. In one aspect, the nanoparticle composition can be administered at a dosage of from about 0.5 ng per kg of subject body weight to about 5 ng per kg of subject body weight, or at about 0.5, 1, 1.5, 2, 2.5, 3, 3.5, 4, 4.5, or about 5 ng per kg of subject body weight, or a combination of any of the foregoing values, or a range encompassing any of the foregoing values, where any of the foregoing values can be an upper or a lower endpoint of the range.

In one aspect, performing the method increases a level of at least one marker for cardiomyocyte cell proliferation, such as, for example, antigen KI-67 (Ki67) or histone 3 phosphorylation (PH3), in a cell or tissue relative to the level of the at least one marker prior to performing the method.

In any of these aspects, the at least one cardiac function or property can be replacement of a myocardial scar with functional contractile tissue, cardiomyocyte renewal, cardiomyocyte cell-cycle activity, reduction in infarct size, reduction in infarct mass, a decline in cardiomyocyte apoptosis, angiogenesis, left ventricular ejection fraction (LVEF), left ventricular fractional shortening (LVFS), end-diastolic volume, end-systolic volume, stroke volume, or any combination thereof.

Further in this aspect, infarct size can be 15% or less of a ratio of an infarct area to a total left ventricular surface area, 4 weeks after performing the method. In another aspect, LVEF can be at least 40%, 4 weeks after performing the method. In still another aspect, LVFS can be at least 20%, 4 weeks after performing the method. In any of these aspects, the heart damage can be a result of myocardial infarction, infection, chemotherapy, heavy alcohol use, cardiac surgery, or any combination thereof.

In one aspect, and without wishing to be bound by theory, when growth factors or small-molecule inhibitors are loaded into PLGA nanoparticles (NPs), the chemicals are slowly released from the NPs over a period of several weeks. In one aspect, the nanoparticle composition is active at a target site for a period of at least 4 weeks. Further in this aspect, disclosed herein are methods of encapsulating TT-10 into PLGA NPs before administration as well as methods of treating MI using the encapsulated TT-10. In a further aspect, the disclosed methods can extend the duration of TT-10 delivery and improve the potency of TT-10 for treatment of MI.

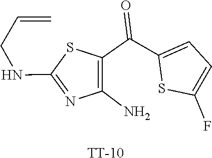 [SMILES: C=CCNc1nc(N)c(C(=O)c2ccc(F)s2)s1 ; "TT-10"]

In one aspect, TT-10 is also known as (2-(allylamino)-4-aminothiazol-5-yl)(5-fluorothiophen-2-yl)methanone.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a nanoparticle,” “an active agent,” or “a cause of cardiac cell damage,” includes, but is not limited to, mixtures or combinations of two or more such nanoparticles, active agents, or causes of cardiac cell damage, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravitreal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be therapeutic in terms of a partial or complete cure of a disease, condition, symptom, or adverse effect attributed to the disease, disorder, or condition. The term “treatment” as used herein can include any treatment of damage to the heart in a subject, particularly a human. As used herein, the term “treating”, can include inhibiting the disease, disorder, or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease.

“Left ventricular ejection fraction” or “LVEF” as used herein refers to the amount of oxygen-rich blood that is pumped out of the left ventricle with each contraction (“stroke volume,” or LVSV) relative to the total amount of blood in the ventricle (“end diastolic volume” or LVEDV). LVEF can be used as a measure of severity of dysfunction on the left side of the heart and is calculated according to the following equation:

$$L V E F = \frac{L V S V}{L V E D V} \times 100$$

“Left ventricular fractional shortening” as used herein refers to the percentage of size differences of the left ventricle as a parameter of how well the left ventricle is contracting and reduces size during systole, and can be calculated using the following equation, where LVESD is the left-ventricular end-systolic diameter and LVEDD is the left ventricular end-diastolic diameter:

$$L V F S = \frac{( L V E D D - L V E S D )}{L V E D D} \times 100$$

“End systolic volume” or “ESV” refers to the amount of blood remaining the ventricle at the end of systole, after the heart has contracted.

“Infarct size” as used herein refers to the ratio of the scar area to the total left ventricular surface area and expressed as a percentage.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration parenterally, paracancerally, intramuscularly, intravenously, intraperitoneally, and/or intraventricularly.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets, or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure can be suitable for injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.

Aspect 1. A nanoparticle composition comprising:
(a) TT-10 or a pharmaceutically acceptable salt thereof and;
(b) nanoparticles comprising at least one polymer or copolymer.

Aspect 2. The nanoparticle composition of aspect 1, wherein the at least one polymer or copolymer is biocompatible.

Aspect 3. The nanoparticle composition of aspect 1, wherein the at least one polymer or copolymer comprises poly-lactic-co-glycolic acid (PLGA).

Aspect 4. The nanoparticle composition of aspect 3, wherein the PLGA has a molecular weight of from about 7000 to about 120,000 Da.

Aspect 5. The nanoparticle composition of aspect 3, wherein the PLGA has a ratio of lactide to glycolide of from about 3:1 to about 1:1.

Aspect 6. The nanoparticle composition of aspect 1, wherein the nanoparticles encapsulate the TT-10.

Aspect 7. The nanoparticle composition of aspect 1, wherein the nanoparticles have an average diameter of from about 75 nm to about 250 nm.

Aspect 8. The nanoparticle composition of aspect 1, wherein the nanoparticle composition comprises from about 2 to about 100 μM of TT-10 or the pharmaceutically acceptable salt thereof.

Aspect 9. A method for improving at least one cardiac function or property following heart damage in a subject, the method comprising administering the nanoparticle composition of aspect 1 to the subject.

Aspect 10. The method of aspect 9, wherein performing the method increases a level of at least one marker for cardiomyocyte cell proliferation in a cell or tissue relative to the level of the at least one marker prior to performing the method.

Aspect 11. The method of aspect 9, wherein the at least one marker comprises antigen KI-67 (Ki67), histone 3 phosphorylation (PH3), or any combination thereof.

Aspect 12. The method of aspect 9, wherein the subject is a human.

Aspect 13. The method of aspect 9, wherein administering the nanoparticle composition comprises intraperitoneal injection, oral administration, parenteral injection, intramuscular injection, intravenous administration, or any combination thereof.

Aspect 14. The method of aspect 9, wherein the nanoparticle composition is administered at a dosage of from about 0.5 to about 5 ng per kg of body weight of the subject.

Aspect 15. The method of aspect 9, wherein the at least one cardiac function or property comprises replacement of a myocardial scar with functional contractile tissue, cardiomyocyte renewal, cardiomyocyte cell-cycle activity, reduction in infarct size, reduction in infarct mass, a decline in cardiomyocyte apoptosis, angiogenesis, left ventricular ejection fraction (LVEF), left ventricular fractional shortening (LVFS), end-diastolic volume, end-systolic volume, stroke volume, or any combination thereof.

Aspect 16. The method of aspect 15, wherein infarct size comprises 15% or less of a ratio of an infarct area to a total left ventricular surface area, 4 weeks after performing the method.

Aspect 17. The method of aspect 15, wherein LVEF is at least 40%, 4 weeks after performing the method.

Aspect 18. The method of aspect 15, wherein LFVS is at least 20%, 4 weeks after performing the method.

Aspect 19. The method of aspect 9, wherein the heart damage is a result of myocardial infarction, infection, chemotherapy, heavy alcohol use, cardiac surgery, or any combination thereof.

Aspect 20. The method of aspect 9, wherein the nanoparticle composition is active at a target site for a period of at least 4 weeks.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Role of TT-10 in Myocardial Regeneration

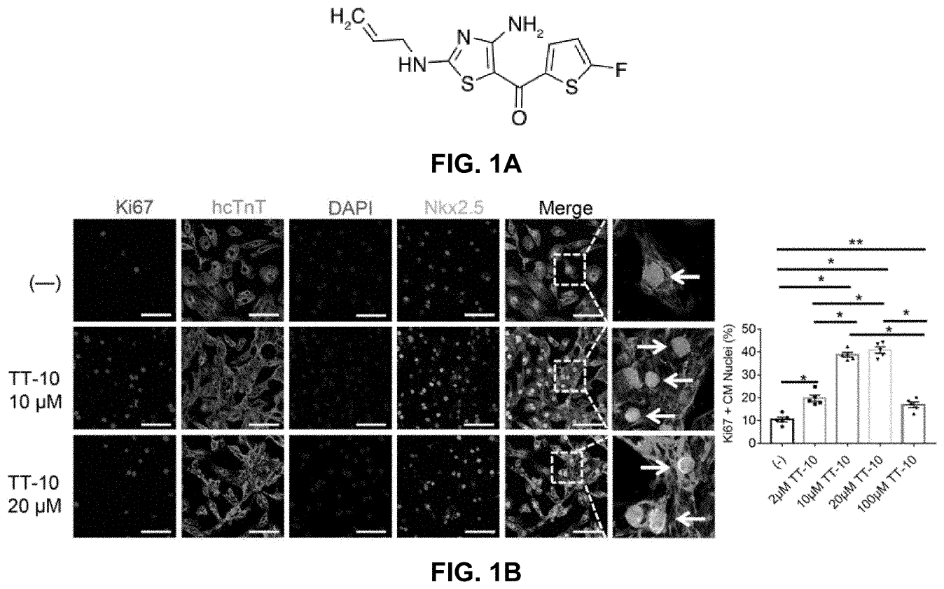

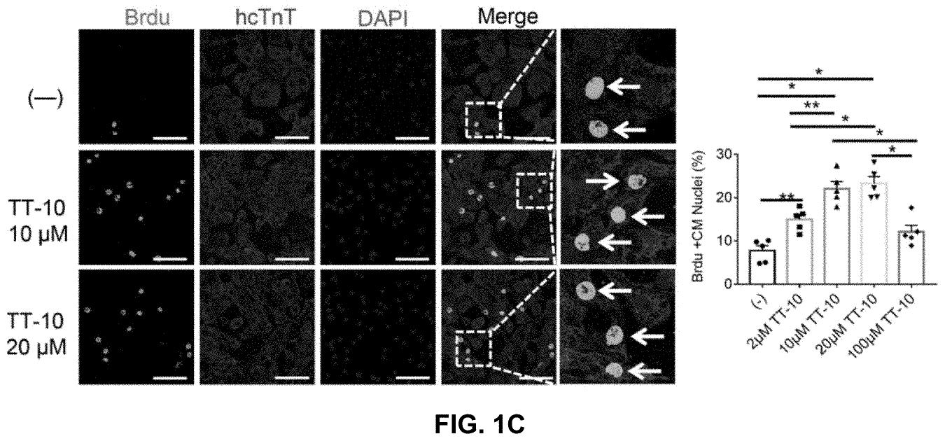

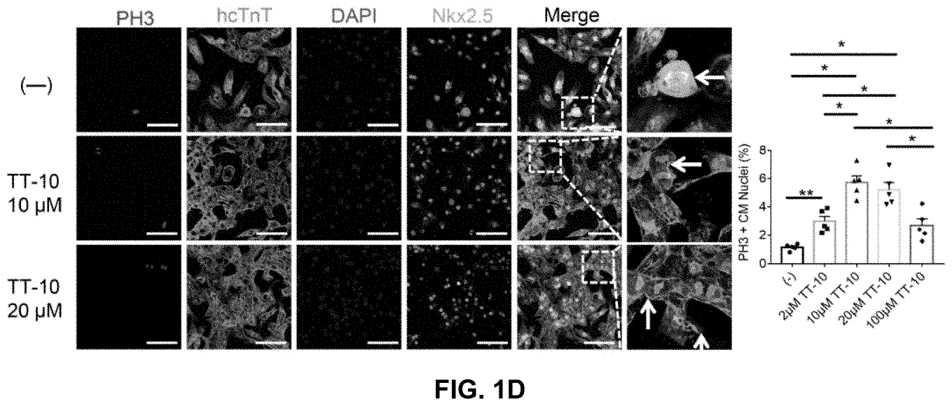

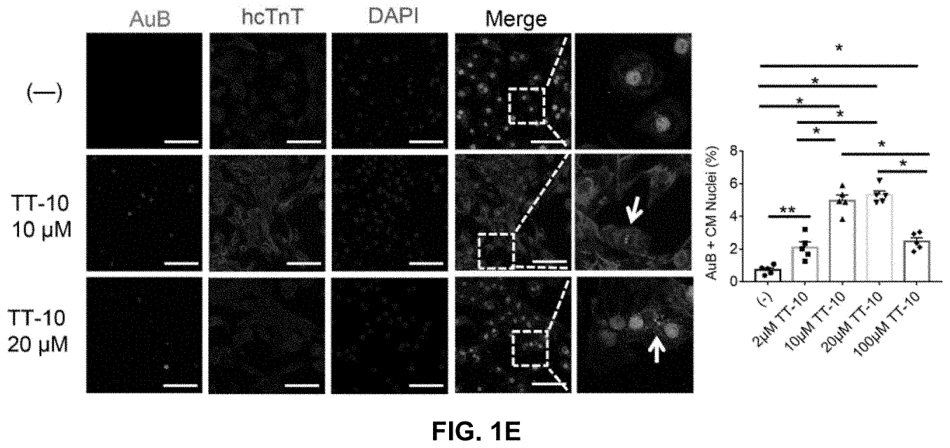

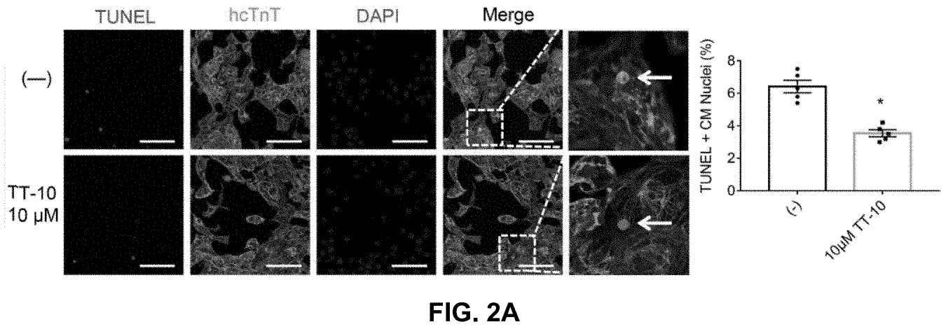

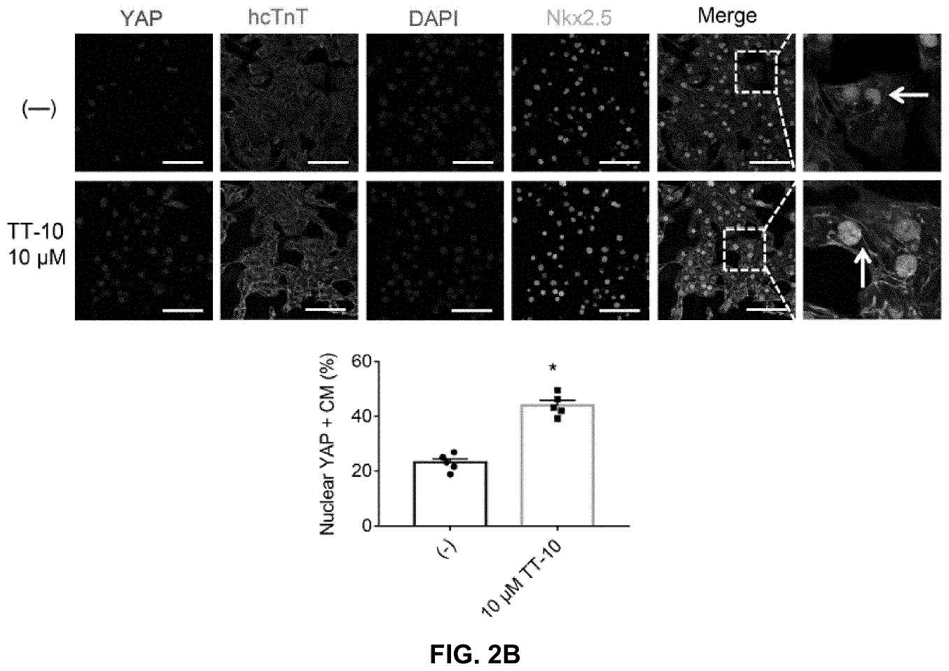

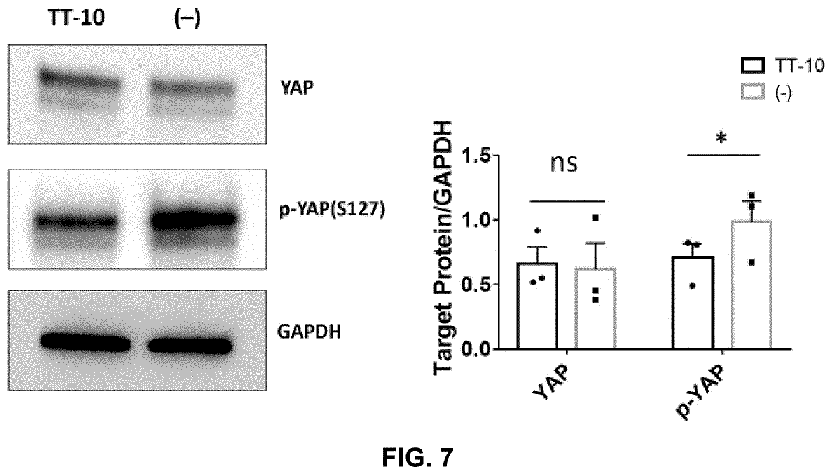

TT-10 promoted cell cycle activity, reduced apoptosis, and upregulated Yap signaling in cultured cardiomyocytes. The effect of TT-10 (FIG. 1A) on cardiomyocyte cell cycle activity and proliferation was determined by culturing human induced pluripotent stem cell-derived cardiomyocytes (hiPSC-CMs) in varying concentrations of TT-10 (0, 2 μM, 10 μM, 20 μM or 100 μM) for 48 hours and then evaluating the presence of markers for cell proliferation (Ki67 expression, FIG. 1B), S phase (BrdU incorporation, FIG. 1C), and M phase (histone 3 phosphorylation [PH3], FIG. 1D) of the cell cycle and for cytokinesis (Aurora B, FIG. 1E). When visualized via immunofluorescence, the proportion of cells displaying each of the 4 markers increased as the TT-10 concentration was raised from 0 to 2 μM, peaked at 10 to 20 μM TT-10, and then declined at 100 μM TT-10. TT-10 treatment (10 μM) also significantly reduced measures of hiPSC-CM apoptosis (TUNEL staining, FIG. 2A) and significantly increased the proportion of hiPSC-CMs with Yap-positive nuclei (FIG. 2B), which is consistent with the role of Hippo/Yap signaling in cardiac regeneration. The effect of TT-10 on YAP pathway activity was also evaluated via Western blot (FIG. 7): total Yap levels in hiPSC-CMs treated with or without TT-10 were similar, but phosphorylated Yap (p-Yap) was less abundant in TT-10-treated cells. Thus, because p-Yap is sequestered and degraded in the cytosol, while unphosphorylated Yap translocates to the nucleus where it induces the expression of genes that regulate cell proliferation and survival, these results suggest that the effect of TT-10 treatment on cell cycle progression and apoptosis in cultured cardiomyocytes was likely mediated, at least in part, by increases in Yap signaling.

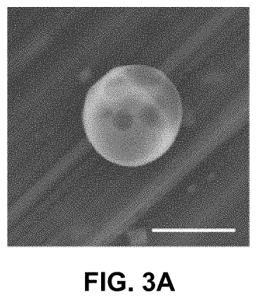

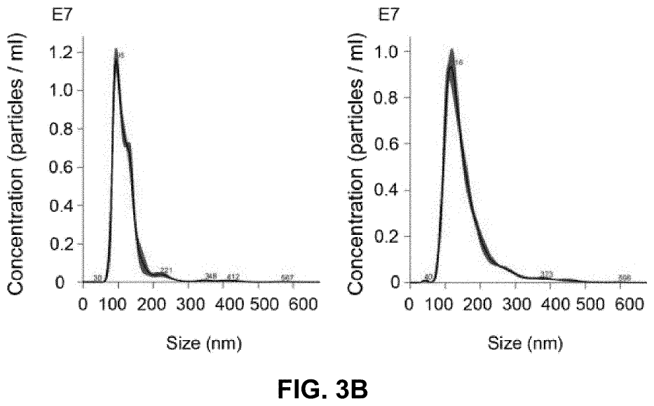

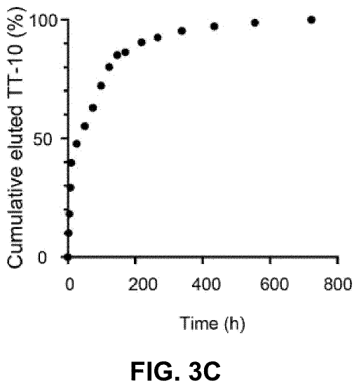

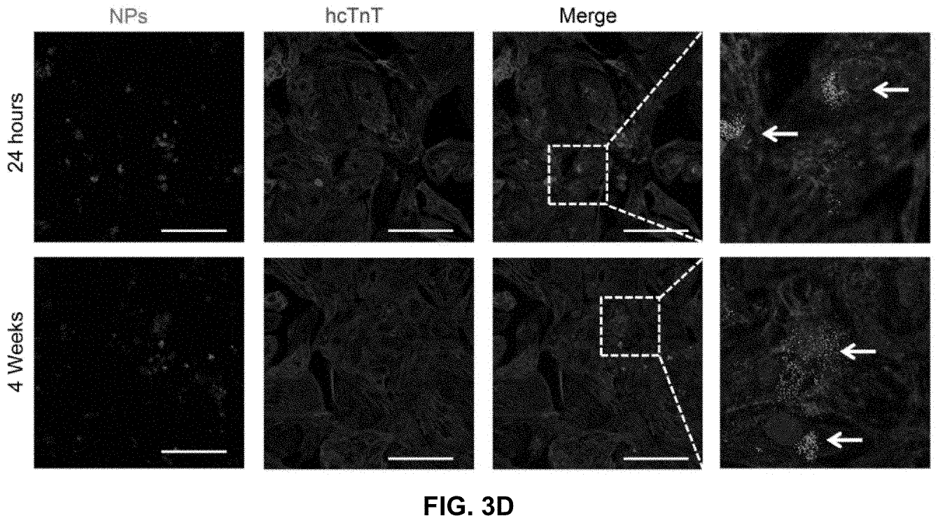

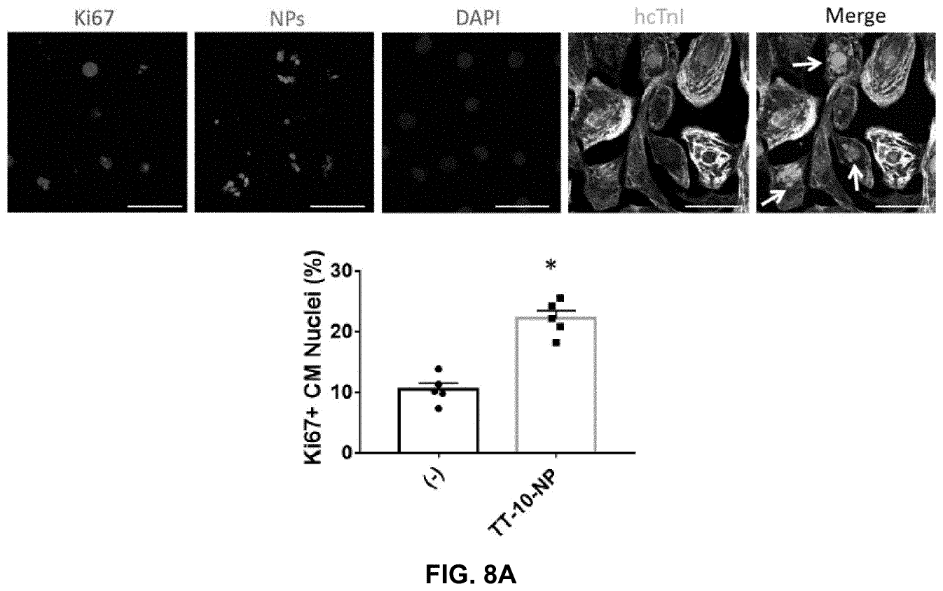

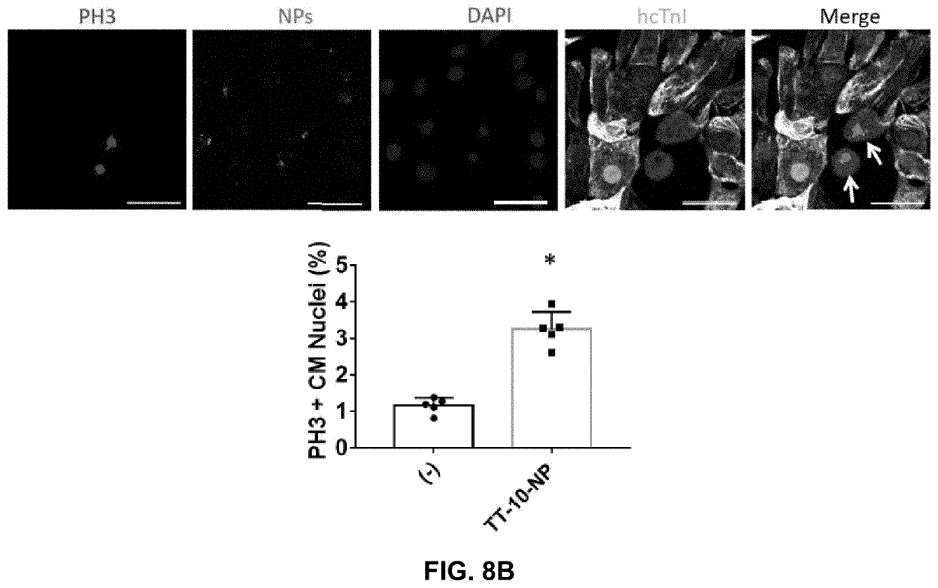

NP-mediated delivery increased the potency of TT-10 for myocardial repair in a mouse MI model. MI was induced via permanent ligation of the left anterior descending (LAD) coronary artery, and then the animals were randomly assigned to treatment with Dulbecco's PBS (DPBS), empty PLGA NPs (Empty-NPs), TT-10 solution (TT-10-SOL), or TT-10-loaded PLGA NPs (TT-10-NPs). A fifth group of animals (the sham group) underwent all surgical procedures for MI induction except arterial ligation. Before TT-10 administration, the mean diameters of the Empty- and TT-10-NPs were 123.3±1.7 nm and 155.4±0.7 nm, respectively (FIGS. 3A-3B). Loading efficiency was found to be 2.50±0.08 μg of TT-10 per mg PLGA NPs. And when the TT-10-NPs (1 mg) were incubated in DPBS (1 mL), 47% of the encapsulated TT-10 was released during the first day and 90% was released by day 9 (FIG. 3C). TT-10-NPs also significantly increased the expression of proliferation markers in cultured hiPSC-CMs (FIGS. 8A-8B), and when hiPSC-CMs were cultured with PLGA-NPs that had been loaded with a fluorescent dye (coumarin-6), immunofluorescence images confirmed that the NPs were taken up by the cells (FIG. 3D).

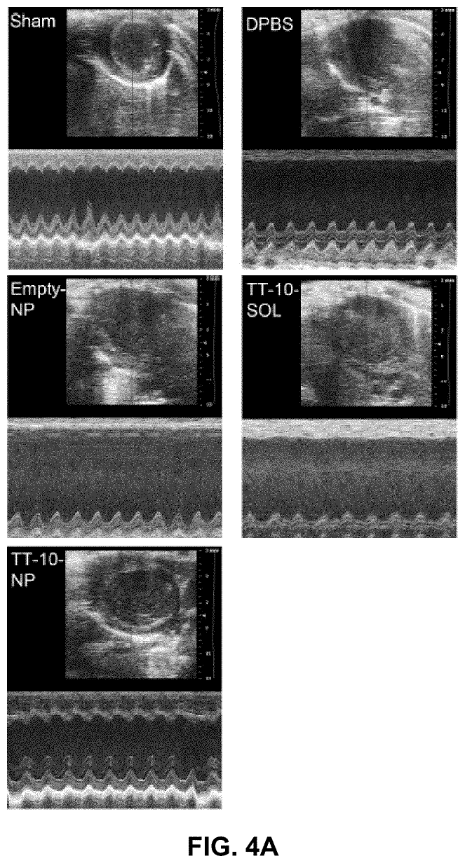

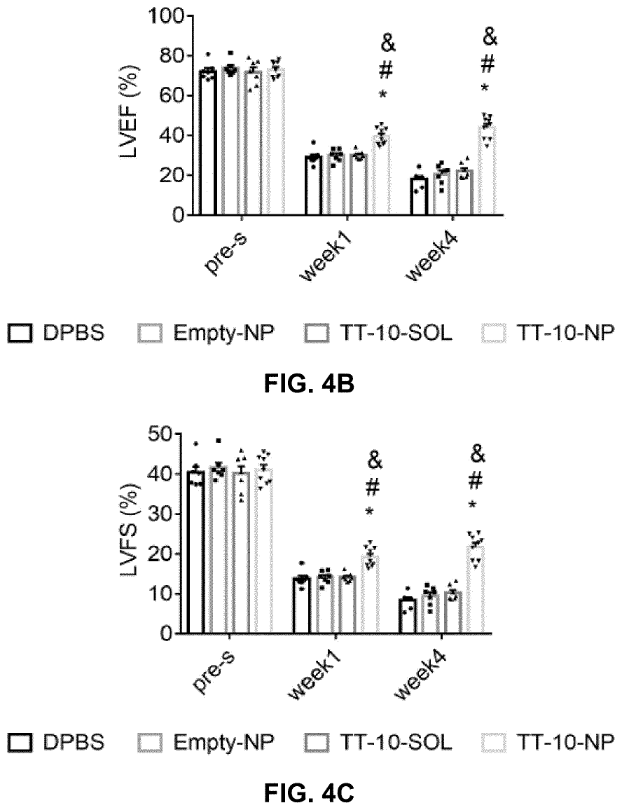

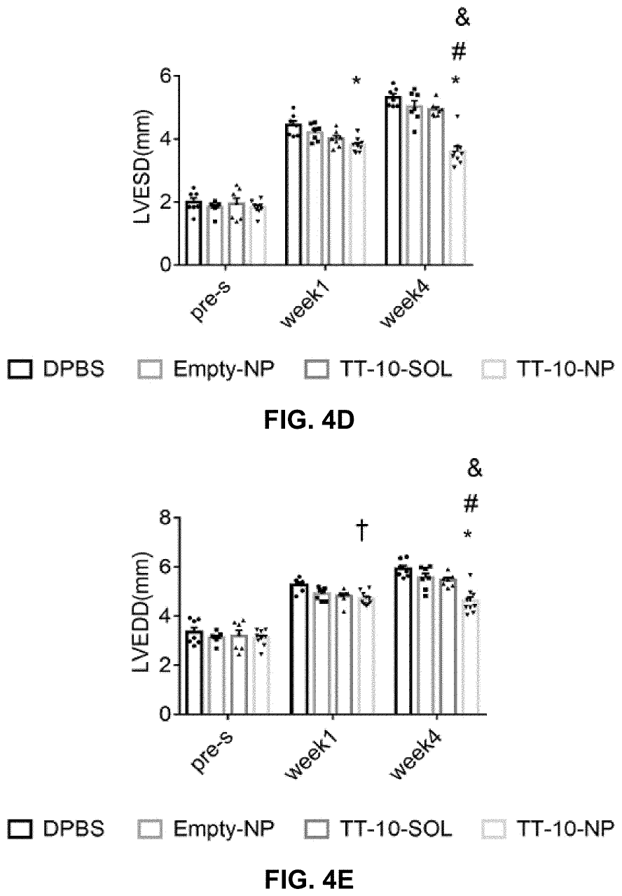

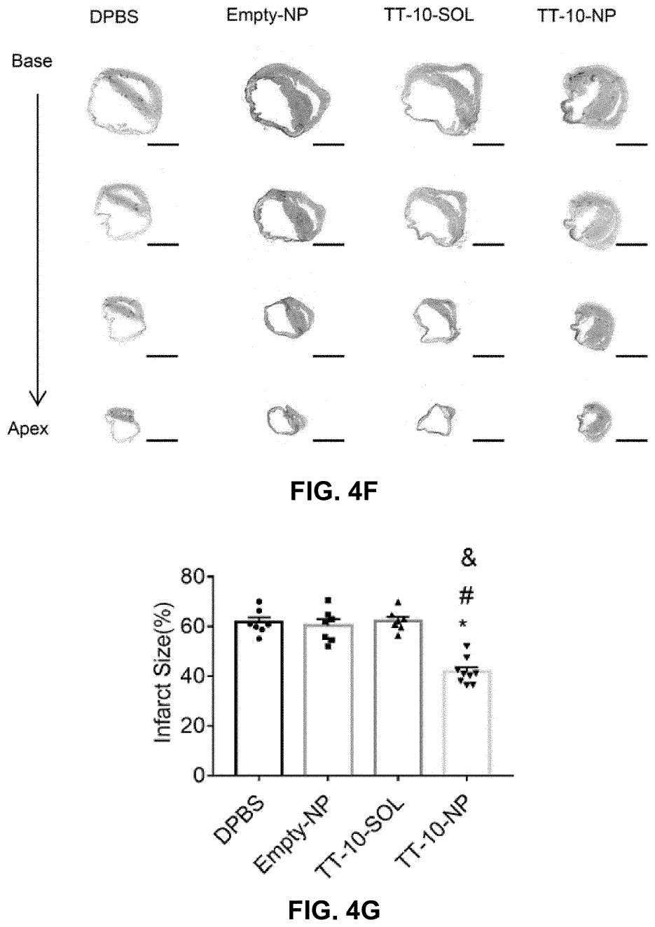

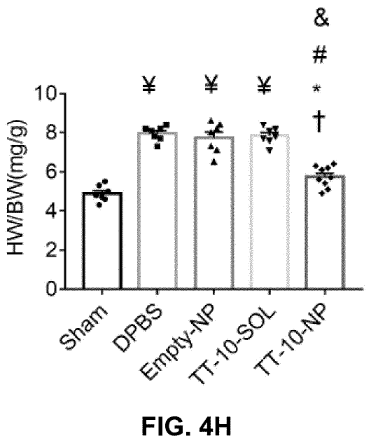

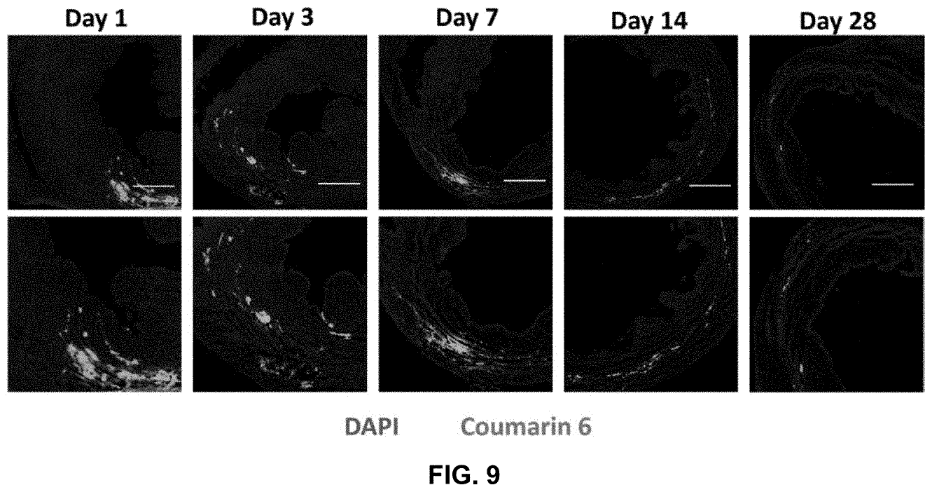

Four mice died from MI induction. In the surviving animals, echocardiographic (FIG. 4A) assessments of left ventricular (LV) ejection fraction (LVEF; FIG. 4B), LV fractional shortening (LVFS; FIG. 4C), LV end-systolic diameter (LVESD; FIG. 4D), and LV end-diastolic diameter (LVEDD; FIG. 4E) did not differ significantly among the DPBS, Empty-NP, and TT-10-SOL treatment groups at week 1 or week 4 after treatment; however, ejection fraction and fractional shortening were significantly greater in TT-10-NP animals at both time points, while end-systolic diameter and end-diastolic diameter were significantly lower at week 4, than in any other group that underwent MI induction. Measurements in animals treated with DPBS, Empty-NPs, or TT-10-SOL also appeared to worsen from week 1 to week 4, while measurements in the TT-10-NP group tended to remain stable (or improve slightly) over the same period. Furthermore, analyses of hearts explanted at week 4 indicated that neither the size of the infarct (FIGS. 4F-4G) nor the heart weight/bodyweight ratio (FIG. 4H) differed significantly among the DPBS, Empty-NP, and TT-10-SOL treatment groups, but both measurements were significantly lower after treatment with TT-10-NP than in any of the other experimental treatment groups. Notably, when NPs were loaded with both TT-10 and coumarin-6 before administration to infarcted mouse hearts, immunofluorescence images of tissues from the border zone (BZ) indicated that most (˜70%) of the NPs were degraded by week 1 and a small proportion (<1%) remained detectable through week 4 (FIG. 9).

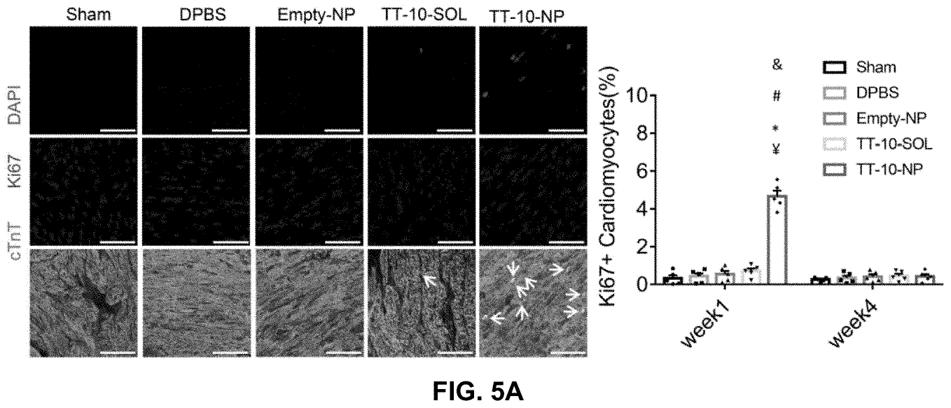

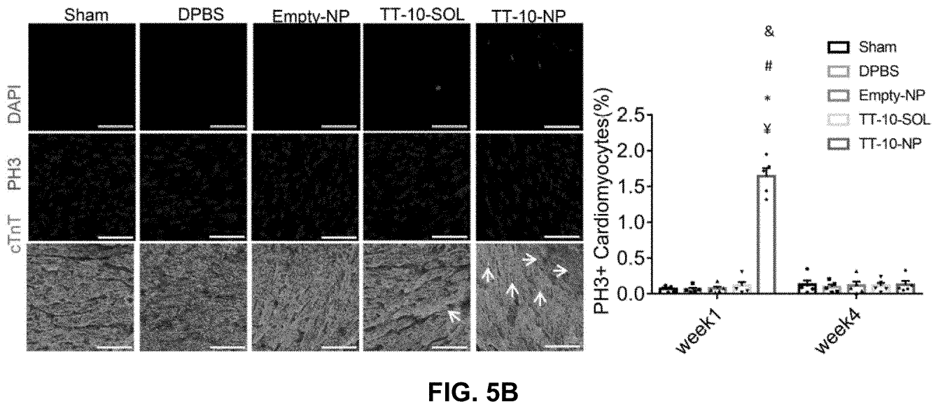

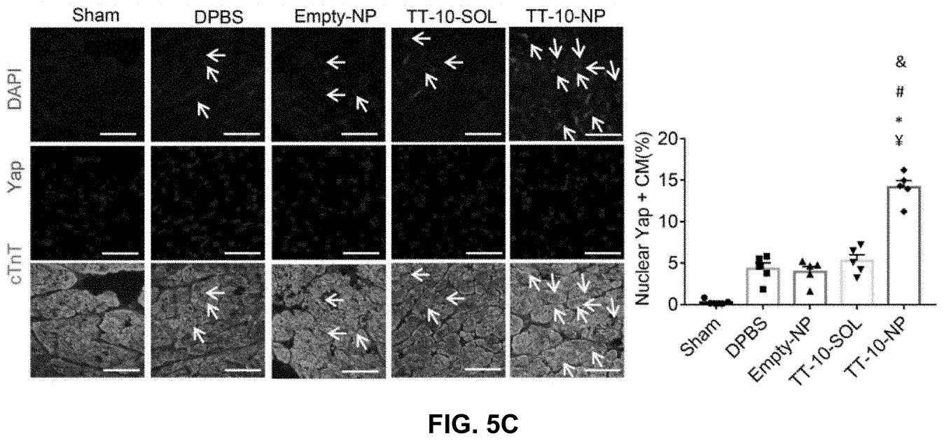

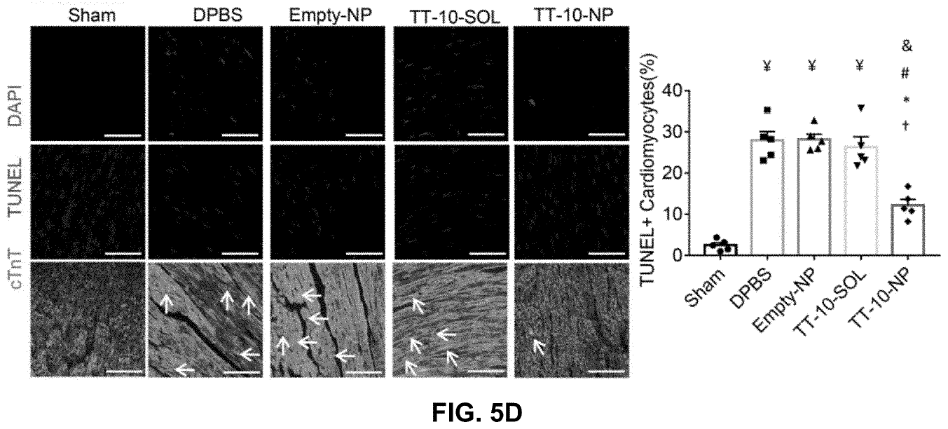

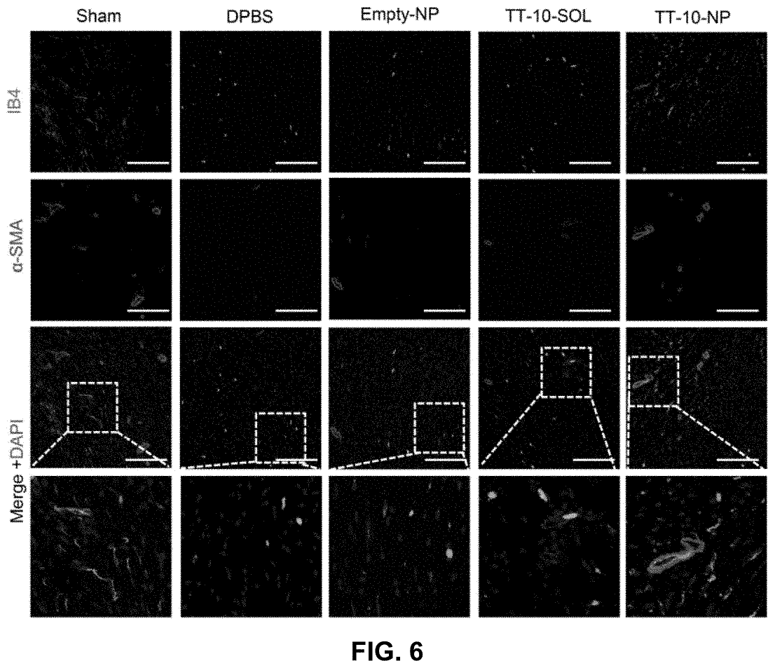

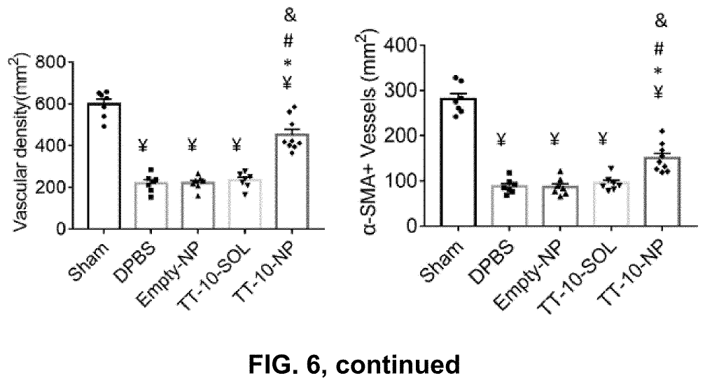

TT-10-NP administration after MI increased cardiomyocyte proliferation, reduced cardiomyocyte apoptosis, and improved vascularity in the peri-infarct region. To determine whether the improvements in myocardial recovery observed in TT-10-NP mice were at least partially attributable to the activation of Hippo/Yap signaling and cardiomyocyte proliferation, subsets of mice were sacrificed 1 week after MI induction, and then cardiomyocytes expressing Ki67 (FIG. 5A), PH3 (FIG. 5B), and nuclear Yap (FIG. 5C) were quantified via immunofluorescence in BZ heart sections. All 3 markers were dramatically more common in BZ cardiomyocytes from TT-10-NP animals than in BZ cardiomyocytes from any other group; however, Ki67 and PH3 fluorescence were nearly undetectable in BZ sections from TT-10-NP mice sacrificed 4 weeks after injury and treatment, likely because cardiomyocytes that began actively cycling in response to TT-10-NP administration exited the cell cycle as the NPs degraded and TT-10 abundance declined. Furthermore, although apoptotic cardiomyocytes were significantly more common in BZ sections obtained from all groups at 72 hours after MI induction than in the corresponding region of hearts from sham animals, measurements were significantly lower in TT-10-NP animals than in the DPBS, Empty-NP, or TT-10-SOL treatment groups (FIG. 5D). TT-10-NP administration also appeared to promote angiogenesis, because vascular structures that stained positively with the endothelial marker isolectin B4 or for the expression of α-smooth muscle actin were significantly more numerous 4 weeks after MI, and treatment in BZ sections obtained from TT-10-NP mice had the greatest number of positive vascular structures compared with those from animals in any other group that underwent MI induction (FIG. 6). Thus, TT-10-NP administration appeared to improve recovery from MI by promoting angiogenesis as well as cardiomyocyte proliferation and survival.

### Example 2: Discussion

The primary mechanism of cardiomyocyte renewal both for maintaining cardiac homeostasis and during recovery from myocardial injury appears to be the proliferation of preexisting cardiomyocytes. However, mammalian cardiomyocytes undergo cell cycle arrest during the perinatal period, and the residual proliferative capacity of adult cardiomyocytes is far too low to regenerate more than a small fraction of the cardiomyocytes that are lost to MI. Thus, therapies targeting the mechanisms that regulate cell cycle activity may be among the most useful strategies for replacing the myocardial scar with functional contractile tissue, and because this approach mimics the endogenous mechanism of cardiomyocyte renewal, the newly generated cells are likely to be better integrated with the native tissue than exogenously administered cells or engineered tissues.

The Hippo signaling pathway impedes proliferation by sequestering YAP and TAZ in the cytosol. Once activated, YAP and TAZ translocate to the nucleus, where they interact primarily with transcription factors of the TEA domain (TEAD) family and the YAP/TAZ-TEAD complex induces the expression of genes that control cell proliferation and apoptosis. TT-10 is a fluorine substituent of TAZ-12; results from large-scale, cell-based screenings of pharmacological small molecules indicated that TT-10 promoted both cardiomyocyte survival and proliferation. Furthermore, when intraperitoneal injections of TT-10 were administered to mice after experimentally induced MI, the treatment promoted cardiomyocyte cell cycle activity and was associated with declines in infarct size. Measures of cardiac function were also significantly better in TT-10-treated animals than in animals administered the delivery vehicle but declined over time in both groups.

Here, PLGA-NPs are used for TT-10 administration, which is more efficient than conventional systemic delivery and, consequently, could enable patients to be treated with lower drug doses, thereby reducing the risk of treatment-related adverse events as well as the demand for largescale TT-10 manufacturing protocols. The beneficial effects associated with free (unencapsulated) TT-10 administration in Hara et al. were achieved with a dose of approximately 250 μg/mouse (i.e., 10 mg/kg×˜25 g/mouse), which is more than 3700-fold greater than the dose administered to animals in the TT-10-SOL and TT-10-NP treatment groups (67.2 ng). Although it is difficult to compare the local accumulated concentration of TT-10 in myocardium between the disclosed TT-10-NPs and Hara's study, it is expected that an enhanced and a long-lasting effect can be achieved by the TT-10-NP delivery approach based on the prolonged release curve (FIG. 3C). This low dose and slow and longer release feature likely contribute to the dramatic increase in efficiency associated with NP-mediated drug administration.

Our results indicated that targeted PLGA-NP-mediated TT-10 delivery improved both the potency and durability of the benefits associated with TT-10 administration: infarct sizes were significantly smaller (by ˜20%), and cardiac performance was significantly better, in animals treated with TT-10-NPs than those in the TT-10-SOL, Empty-NP, or DPBS treatment groups. While functional parameters remained stable from weeks 1 to 4 in TT-10-NP animals, measurements in the other 3 groups that underwent MI induction worsened over the same period. TT-10-NP administration was also associated with increases in the frequency of Ki67, PH3, and nuclear Yap expression among cardiomyocytes 1 week after treatment, as well as with declines in cardiomyocyte apoptosis on day 3, which was consistent with observations in cultured hiPSC-CMs and suggests that the benefits of treatment evolved, at least in part, via the Yap/TAZ-induced activation of cell cycle progression and cardioprotective mechanisms. However, proliferating cardiomyocytes were nearly undetectable 4 weeks after TT-10-NP delivery, which may be beneficial, because proliferating cardiomyocytes are typically immature and, consequently, could increase the risk of arrhythmia and disrupt contractile activity. TT-10-NP administration also promoted angiogenesis in the peri-infarct region, which has not been previously reported, and Yap/TAZ signaling can be activated by hypoxia in some cancers, as well as extracellular matrix proteins (e.g., periostin), growth factors, and microRNAs, all of which have been linked to cardiomyocyte cell cycle activity during development or under disease conditions. Thus, cardiomyocyte cell cycle activity in mammalian hearts appears to be governed by a broad range of physiological conditions and signaling mechanisms. The cytoprotective effects of TT-10-NP administration likely occurred during the first few days after administration, when cardiomyocyte apoptosis peaks, and may have been mediated by declines in the abundance of ROS and ROS-induced DNA damage.

Studies of fluorine-containing pharmaceuticals, such as TT-10, are becoming increasingly common, because these pharmaceuticals are metabolically stable and, consequently, have high bioavailability in vivo. However, their efficacy for treatment of cardiovascular disorders is hampered by poor retention at the site of administration. Thus, these results suggest that PLGA NPs could be used to improve the efficiency of treatment administration for numerous cardiovascular drugs. Furthermore, although the animals in the current investigation were treated with TT-10-NPs via direct intramyocardial injections during open-chest surgery, PLGA NPs are fully compatible with less invasive clinical delivery methods, such as catheter-based or echo-guided transthoracic myocardial injection.

In summary, the results from this investigation in a mouse MI model demonstrate that measures of cardiac function and infarct size are significantly better when the animals receive intramyocardial injections of TT-10-loaded NPs rather than TT-10-SOL. Thus, PLGA-NP-mediated delivery appears to enhance the potency of TT-10, which may also reduce the occurrence of treatment-related side effects and other safety concerns by enabling the drug to be administered at lower doses. Collectively, these observations support the continued development of TT-10-NPs for the treatment of cardiac disease and provide proof of concept for the PLGA-NP-mediated delivery of other therapeutic agents.

### Example 3: Methods

Preparation and characterization of PLGA NPs. PLGA NPs were prepared via a single-emulsion (oil/water phase) technique as described previously. Briefly, a solution of PLGA (100 mg) in dichloromethane (5 mL) with or without TT-10 (300 μL at 2 mg/mL) or coumarin-6 (1 mg) was ultrasonicated at 40% amplitude in 40-second intervals with 20-second pauses for a total of 2 minutes. Then, 20 mL of 1% (w/v) dimethylamine borane-water solution was added, and the mixture was ultrasonicated on ice at 40% amplitude in 40-second intervals with 20-second pauses for a total of 2 minutes. The mixture was slowly transferred into 10 mL 4% (w/v) PVA-water solution, added with 30 mL Milli-Q water (MilliporeSigma) into a 100 mL glass beaker, and stirred for 4 hours until the dichloromethane completely evaporated. Then, the solution was centrifuged at 1000 g for 10 minutes to remove bulk aggregates, and the supernatant was centrifuged at 45,000 g for 20 minutes to collect the NPs. The NPs were washed twice by resuspending them in 50 mL Milli-Q water; they were then recollected via centrifugation (45,000 g for 20 minutes) and frozen at −80° C. overnight, lyophilized for 48 hours, and stored at −80° C.

NP size measurements were performed via NP-tracking analysis with a NanoSight NS300 Instrument (NanoSight), and scanning electron microscopy was performed with a Quanta FEG 650 (FEI). For TT-10-loaded NPs, TT-10 release was monitored with a Slide-A-Lyzer MINI Dialysis Device (Thermo Fisher Scientific); the molecular weight cutoff was set at 20,000, and the elution medium consisted of DPBS with 0.1% BSA. Suspensions were incubated at 37° C. with constant shaking (300 rpm), and 14.5 mL samples of the elution medium were withdrawn and replaced at the indicated time points. TT-10 concentrations in the collected samples were measured with a UV/Vis spectrophotometer (NanoDrop, Thermo Fisher Scientific); measurements of TT-10 absorbance (λ=˜200 nm) in samples were compared with a curve constructed from known TT-10 concentrations using the Beer-Lambert Law.

Differentiation of hiPSCs into cardiomyocytes. The hiPSCs were maintained in 6-well plates with mTeSR medium (Stem Cell Technologies) until 80% confluent and then differentiated into hiPSC-CMs. Briefly, the hiPSCs were cultured in RPMI 1640 medium with 2% B27 minus insulin (RB medium; Gibco), 10 μM CHIR99021 (Stem Cell Technologies), and 1 μg/mL insulin (MilliporeSigma) for 24 hours; in RB medium containing 3 μM CHIR99021 for 48 hours; in RB medium containing 10 μM IWR1 (Stem Cell Technologies) for 48 hours; in RB medium for 48 hours; and then in RPMI 1640 medium supplemented with 2% B27 supplement (Gibco). Differentiated hiPSC-CMs were purified via metabolic selection in glucose-free RPMI 1640 medium (Gibco) containing B27 supplement (Gibco) and 4 mM lactate (MilliporeSigma) for 5 days and then maintained in RPMI 1640 medium supplemented with 2% B27 supplement (Gibco). Experiments were conducted 30 days after differentiation was initiated.

Cellular uptake of PLGA NPs. hiPSCs-CMs were seeded onto a Lab-Tek chamber slide system (Nunc, Thermo Fisher Scientific), treated with coumarin-6-loaded NPs (2 μg/mL), incubated at 37° C. and 5% CO₂ for 24 hours; washed twice with DPBS (pH 7.4); and visualized with a fluorescent microscope.

Mouse MI model. Eight- to twelve-week-old male and female C57BL/6 mice (The Jackson Laboratory) were anesthetized with inhaled isoflurane (2%), intubated, and ventilated with 2% isoflurane. A left thoracotomy was performed, and the LAD coronary artery was ligated with an 8 to 0 nonabsorbable suture (24). Animals in the TT-10-NP and Empty-NP groups were treated with PLGA NPs that had or had not, respectively, been loaded with TT-10; animals in the TT-10-SOL group were treated with TT-10-SOL; and animals in the DPBS group were treated with DPBS. The total dosage of TT-10 used in both the TT-10-SOL and TT-10-NP groups was 67.2 ng. For the sham group, a suture was positioned around the LAD coronary artery without ligation. Treatments were administered via intramyocardial injection into 3 sites surrounding the infarct; injections were administered with a modified Hamilton needle, and the total volume of the injection was 24 μL (8 μL/site). Intraperitoneal injections of buprenorphine hydrochloride (0.1 mg/kg, Buprenex, Reckitt Benckiser Pharmaceuticals Inc.) and carprofen (5 mg/kg, Rimadyl, Zoetis) were administered after chest closure for pain control.

Echocardiography. Animals were lightly anesthetized with 1%-1.5% inhaled isoflurane, and heart rates remained stable at 400-500 bpm. Then, B-mode and 2-dimensional M-mode images were obtained from long-axis and short-axis views with a high-resolution microultrasound system (Vevo 2100, Visual-Sonics Inc.). Data were analyzed with Vevo analysis software, and LVEF, LVFS, LVEDD, and LVESD were calculated.

Infarct size. Infarct size was evaluated as described previously. Briefly, hearts were excised, fixed in 4% paraformaldehyde (PFA) for 4 hours, immersed overnight in 30% sucrose at 4° C., embedded in OCT compound, and sectioned at a thickness of 10 μm. Every twentieth section from the base to the apex was fixed in Bouin's solution and stained with Picrosirius Red/Fast Green dyes. Then, images of the stained sections were obtained with an Olympus light microscope and analyzed with NIH ImageJ software. Infarct size was calculated according to the following formula: infarct size=(scar circumferential length×thickness of each of the short axis)/(short-axis LV length×thickness of the short axis)×100%.

Immunofluorescence. For hiPSC-CMs, the cells were immobilized with 4% PFA for 10 minutes; permeabilized with acetone for 1 minute; washed with PBS+0.1% Tween 20 (PBST); blocked with 10% donkey serum (Life Technologies) for 30 minutes; incubated at 4° C. overnight with primary antibodies; and incubated for 40 minutes with fluorophore-linked secondary antibodies; then, nuclei were stained with DAPI at room temperature for 10 minutes. For heart sections, tissues were fixed in ice-cold 4% PFA for 4 hours, immersed in 30% sucrose overnight at 4° C., and cut into 10 μm sections; then, the sections were washed with PBST for 10 minutes, permeabilized with cold acetone for 3 minutes, immobilized with 4% PFA for 10 minutes, blocked with 10% donkey serum for 30 minutes, incubated with primary antibodies at 4° C. overnight, and incubated with fluorophore-linked secondary antibodies for 40 minutes; then, nuclei were stained with DAPI at room temperature for 10 minutes. After staining, analyses were conducted with a fluorescence microscope.

BrdU labeling. hiPSC-CMs were incubated with BrdU (10 μmol) in a humidified atmosphere containing 5% CO2 at 37° C. for 12 hours, fixed with ice-cold 70% ethanol (pH 2.0, 50 mM glycine) for 15 minutes, and then stained with a BrdU Labeling and Detection Kit (Roche Life Science Inc.) as directed by the manufacturer's instructions.

TUNEL. TUNEL staining was performed with an in situ cell death detection kit (Roche). Briefly, cells or heart sections were fixed with 4% PFA at room temperature for 15 minutes, TUNEL-labeled as directed by the manufacturer's instructions, washed with PBST, blocked with 10% donkey serum (Life Technologies) for 30 minutes, incubated with primary antibodies at 4° C. overnight, and incubated with fluorophore-linked secondary antibodies at room temperature for 40 minutes. Then, nuclei were stained with DAPI. Apoptosis was quantified as the proportion of cells that were TUNEL positive.

Western blot. hiPSC-CMs were lysed with M-PER Mammalian Protein Extraction Reagent (Thermo Fisher Scientific) supplemented with Halt Protease and Phosphatase Inhibitor Cocktail (Thermo Fisher Scientific). Extracted protein was quantified by BCA protein assay kit (Thermo Fisher Scientific) and denatured by heating at 95° C. for 10 minutes. Equal quantity of protein were loaded and separated by 4%-15% precast SDS-PAGE gels (Bio-Rad), and then transferred onto a polyvinylidene difluoride membrane (Bio-Rad). The membranes were blocked with 5% nonfat dry milk and incubated at 4° C. overnight with primary antibodies, as described in Antibodies. HRP-conjugated secondary antibody was used at 1:4000 dilution for detection. Finally, the membranes were developed with Immobilon Western Chemiluminescent HRP Substrate (MilliporeSigma). Protein signals were imaged with a Bio-Rad ChemiDoc System and analyzed by ImageJ. The housekeeping protein, GAPDH, was used for normalization.

Antibodies. The following primary antibodies and second antibodies were used in the study: mouse anti-cardiac troponin T monoclonal antibody (1:100, Thermo Fisher Scientific, catalog MAB1874); rabbit anti-cardiac troponin T monoclonal antibody (1:100, Abcam, catalog ab91605); mouse anti-cardiac troponin T monoclonal antibody (1:100, Thermo Fisher Scientific, catalog MS295P1); goat anti-cardiac troponin I polyclonal antibody (1:100, Abcam, catalog ab188877); rabbit anti-Ki67 monoclonal antibody (1:100, Abcam, catalog ab16667); rabbit anti-phospho-histone H3 (Ser10) polyclonal antibody (1:1000, MilliporeSigma, catalog 06-570); mouse anti-Aurora B monoclonal antibody (1:50, BD Biosciences, catalog 611082); goat anti-human NKX2.5 polyclonal antibody (1:25, R&D Systems, Thermo Fisher Scientific, AF2444); rabbit anti-YAP monoclonal antibody (1:100 for immunofluorescence and 1:1000 for Western blot, Cell Signaling Technology, catalog 14074S); rabbit anti-phospho YAP monoclonal antibody (1:1000, Cell Signaling Technology, catalog 13008S); fluorescein-labeled GSL I IB4 (1:10, Vector laboratories, catalog FL-1201); rabbit anti-α smooth muscle actin polyclonal antibody (1:200, Abcam, catalog ab5694); Cy™ 3 donkey anti-rabbit polyclonal antibody (1:200, Jackson ImmunoResearch Laboratory, catalog 711-165-152); Alexa Fluor 488 donkey anti-mouse polyclonal antibody (1:200, Jackson ImmunoResearch Laboratory, catalog 715-545-150); Alexa Fluor 647 donkey anti-goat polyclonal antibody (1:200, Jackson ImmunoResearch Laboratory, catalog 705-605-147); and anti-rabbit HRP linked antibody (1:4000, Cell Signaling Technology, catalog 7074P2).

Statistics. All data are presented as mean±SEM and were evaluated for significance with GraphPad Prism 7 software. Comparisons between 2 groups were conducted with the 2-tailed Student's t test, and comparisons among 3 or more groups were conducted via 1- or 2-way ANOVA with adjustment. P<0.05 was considered statistically significant.

Study approval. All protocols were approved by the Institutional Animal Care and Use Committee of the University of Alabama at Birmingham and were performed in accordance with the Guide for the Care and Use of Laboratory Animals (National Academies Press, 2011).

### Example 4: TT-10-PLGA Nanoparticles in a Pig Model of Acute Myocardial Infarction (AMI)

TT-10-PLGA nanoparticles prepared as disclosed above and administered to Yorkshire pigs following AMI induction.

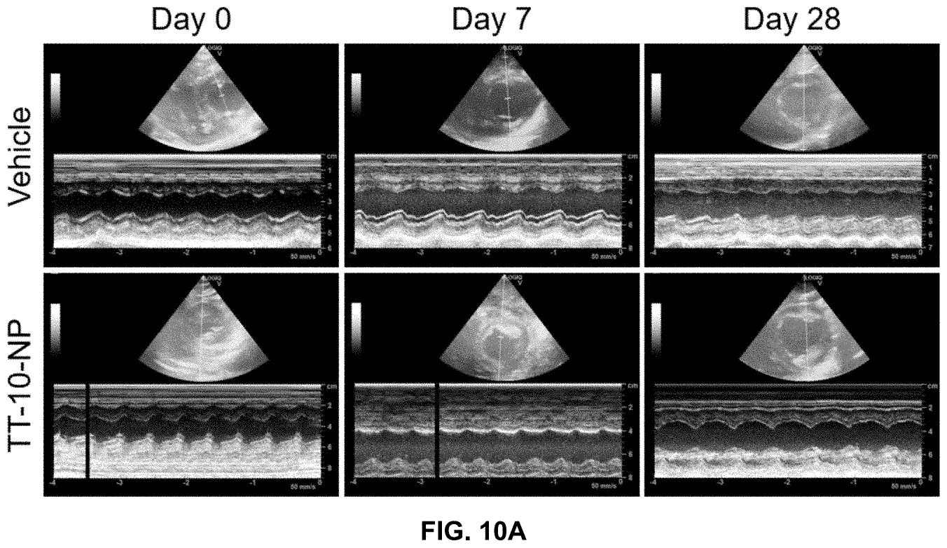

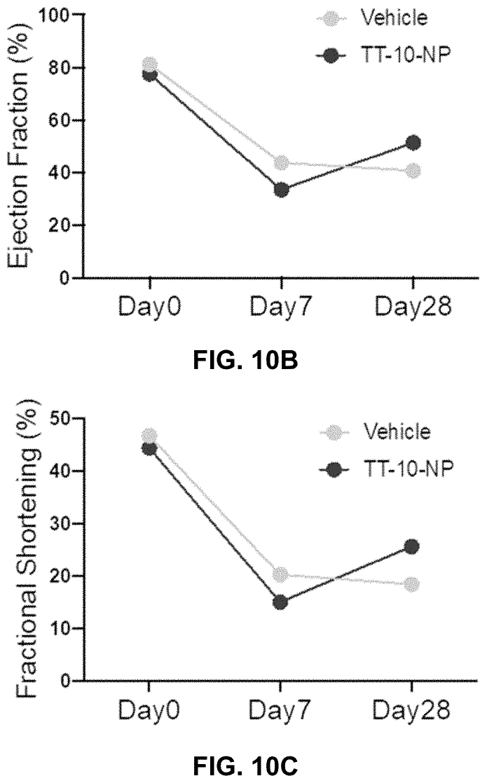

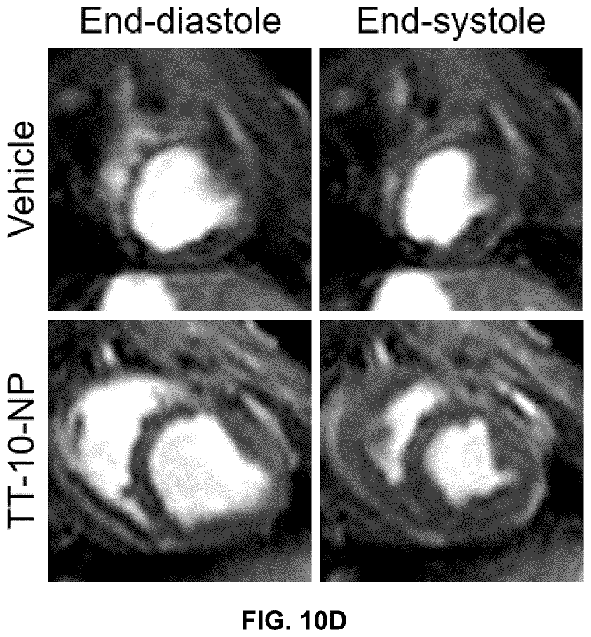

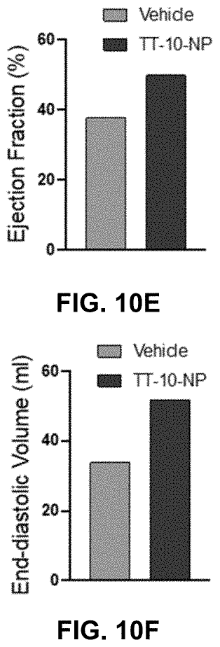

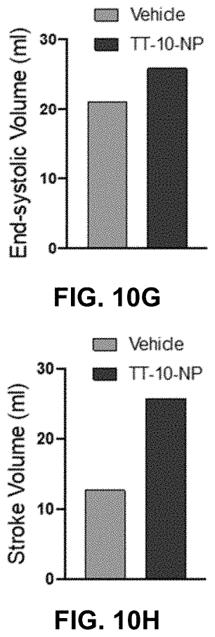

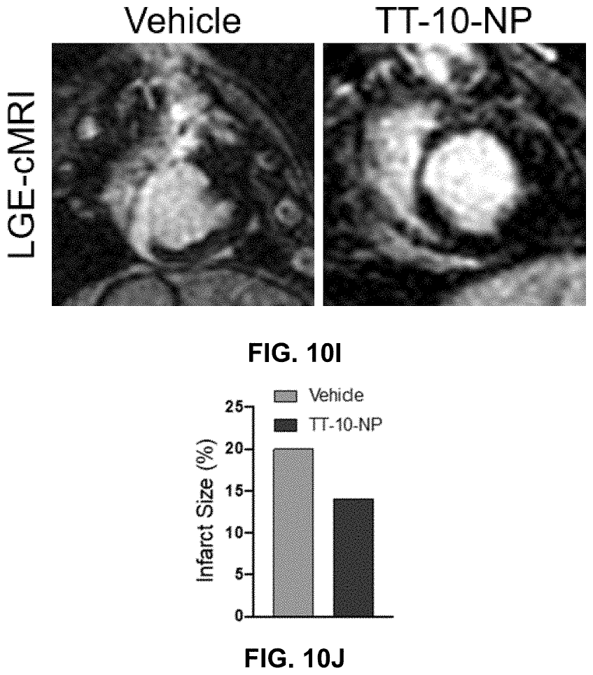

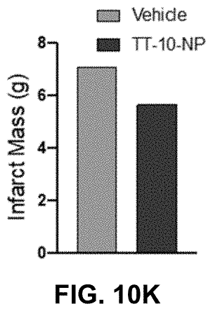

Intramyocardial injections of TT-10-NP improved cardiac function and reduced infarct size in infarcted pig hearts. Acute myocardial infarction was induced by ligating left anterior descending (LAD) coronary artery of Yorkshire pigs (˜15 kg body weight), following by injecting of TT-10-NP (0.5 mg/kg body weight) or equal amount of delivery vehicle (1 mL PBS). (FIG. 10A) Echocardiographic images were obtained for pigs in the Vehicle and TT-10-NP groups before surgery, on Day 7 and Day 28 after AMI induction and used to calculate (FIG. 10B) LVEF and (FIG. 10C) LVFS. (FIGS. 10D-10H) cMRI was performed on Day 28 and used to calculate (FIG. 10E) LVEF, (FIG. 10F) Stroke Volume, (FIG. 10G) End-systolic Volume, and (FIG. 10H) End-diastolic Volume. (FIGS. 10I-10J) LGE-cMRI images were obtained in pigs on Day 28 after MI induction and used to calculate (FIG. 10J) infarct size and (FIG. 10K) infarct mass.

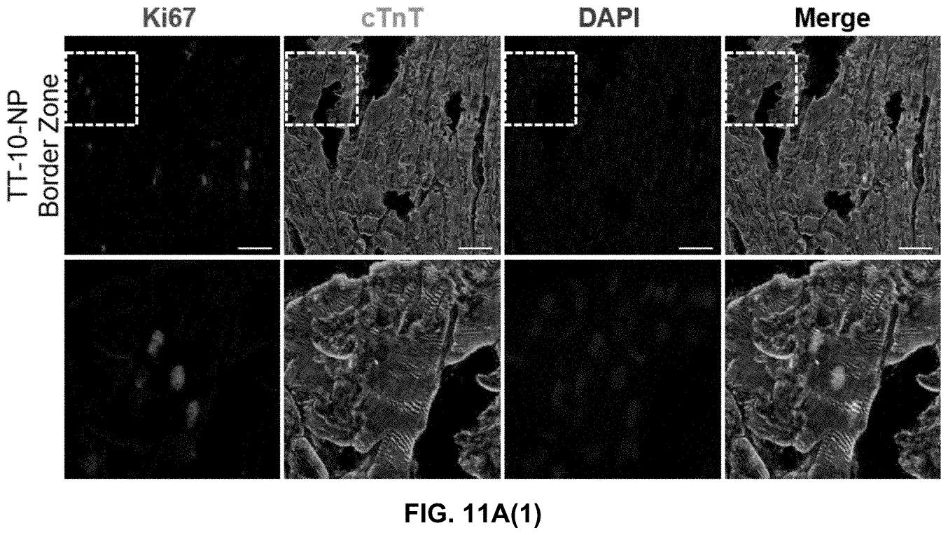

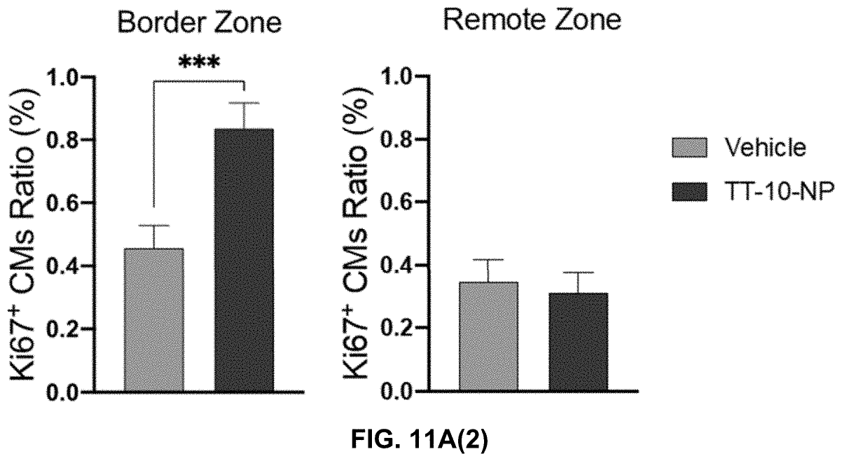

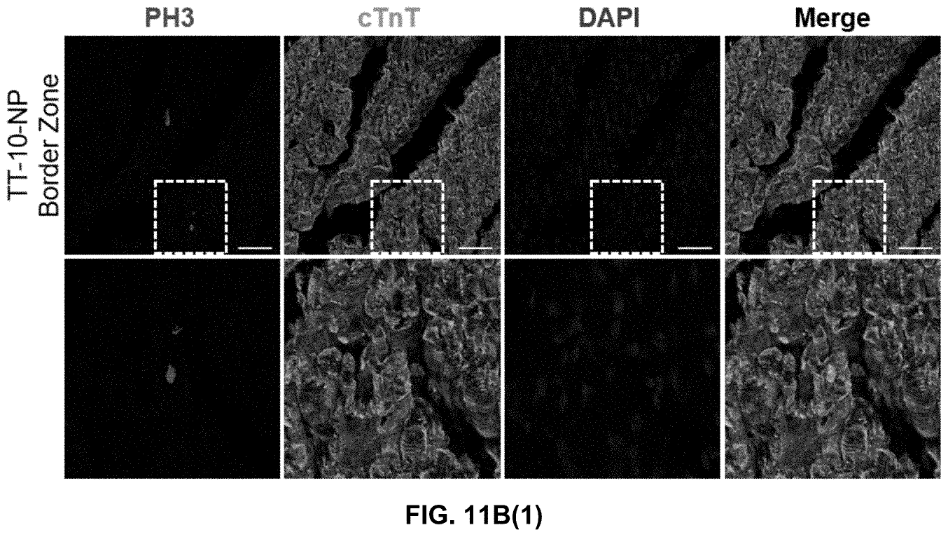

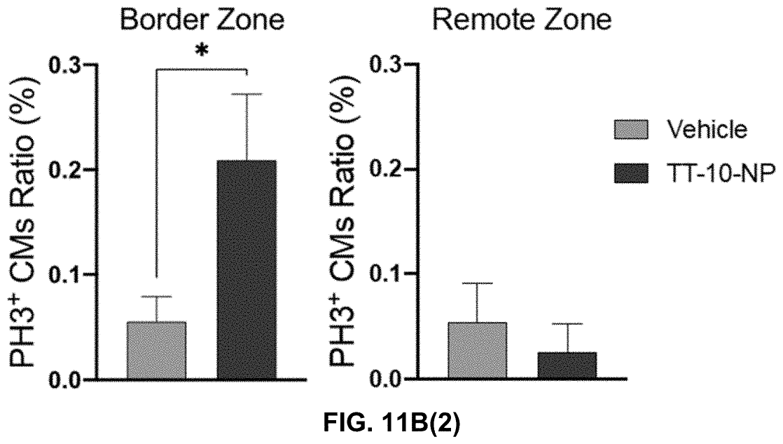

Intramyocardial injections of TT-10-NP promoted cardiomyocyte cell-cycle activity in a pig AMI model. Sections of heart tissue from the border zone and remote zone of the infarct were collected from pigs 28 days after MI and stained for the presence of cTnT and (FIGS. 11A(1)-11A(2)) Ki67 or (FIGS. 11B(1)-11B(2)) PH3; then, cardiomyocyte proliferation and cell-cycle activity were quantified as the percentages of cTnT-positive cells that were also positive for Ki67 and PH3, respectively. Quantified data of Ki67 and PH3 were obtained from 4 sections per cardiac ring, 5 high-power viewing fields per section; 2 border zone cardiac rings and 1 remote cardiac ring were examined. Bar=50 μm. *P<0.05, ***P<0.001.

Statistics. All data are presented as mean±SEM and were evaluated for significance with GraphPad Prism 7 software. Comparisons between 2 groups were conducted with the 2-tailed Student's t test, and comparisons among 3 or more groups were conducted via 1- or 2-way ANOVA with adjustment. P<0.05 was considered statistically significant.

Study approval. All protocols were approved by the Institutional Animal Care and Use Committee of the University of Alabama at Birmingham and were performed in accordance with the Guide for the Care and Use of Laboratory Animals (National Academies Press, 2011).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Bergmann O, et al. Evidence for cardiomyocyte renewal in humans. Science. 2009; 324(5923):98-102.
2. Bersell K, et al. Neuregulin1/ErbB4 signaling induces cardiomyocyte proliferation and repair of heart injury. Cell. 2009; 138(2):257-270.
3. Boopathy G T K, Hong W. Role of hippo pathway-YAP/TAZ signaling in angiogenesis. Front Cell Dev Biol. 2019; 7:49.
4. Engel F B, et al. FGF1/p38 MAP kinase inhibitor therapy induces cardiomyocyte mitosis, reduces scarring, and rescues function after myocardial infarction. Proc Natl Acad Sci USA.
5. Eulalio A, et al. Functional screening identifies miRNAs inducing cardiac regeneration. Nature. 2012; 492(7429):376-381.
6. Fan C, et al. Myocardial protection by nanomaterials formulated with CHIR99021 and FGF1. JCI Insight. 2020; 5(12):e132796.
7. Gabisonia K, et al. MicroRNA therapy stimulates uncontrolled cardiac repair after myocardial infarction in pigs. Nature. 2019; 569(7756):418-422.
8. Hara H, et al. Discovery of a small molecule to increase cardiomyocytes and protect the heart after ischemic injury. JACC Basic Transl Sci. 2018; 3(5):639-653.
9. Heallen T, et al. Hippo pathway inhibits Wnt signaling to restrain cardiomyocyte proliferation and heart size. Science. 2011; 332(6028):458-461.
10. Kuhn B, et al. Periostin induces proliferation of differentiated cardiomyocytes and promotes cardiac repair. Nat Med. 2007; 13(8):962-969.
11. Lin Z, et al. Cardiac-specific YAP activation improves cardiac function and survival in an experimental murine MI model. Circ Res. 2014; 115(3):354-363.
12. Mattapally S, et al. Spheroids of cardiomyocytes derived from human-induced pluripotent stem cells improve recovery from myocardial injury in mice. Am J Physiol Heart Circ Physiol. 2018; 315(2): H327-H339.
13. Muller K, et al. Fluorine in pharmaceuticals: looking beyond intuition. Science. 2007; 317(5846):1881-1886.
14. Nakada Y, et al. Hypoxia induces heart regeneration in adult mice. Nature. 2017; 541(7636):222-227.
15. Oduk Y, et al. VEGF nanoparticles repair the heart after myocardial infarction. Am J Physiol Heart Circ Physiol. 2018; 314(2):H278-H284.
16. Patra J K, et al. Nano based drug delivery systems: recent developments and future prospects. J Nanobiotechnology. 2018; 16(1):71.
17. Porrello E R, et al. Transient regenerative potential of the neonatal mouse heart. Science. 2011; 331(6020):1078-1080.
18. Puente B N, et al. The oxygen-rich postnatal environment induces cardiomyocyte cell-cycle arrest through DNA damage response. Cell. 2014; 157(3):565-579.
19. Senyo S E, et al. Mammalian heart renewal by pre-existing cardiomyocytes. Nature. 2013; 493(7432):433-436.
20. Vilahur G, et al. Molecular and cellular mechanisms involved in cardiac remodeling after acute myocardial infarction. J Mol Cell Cardiol. 2011; 50(3):522-533.
21. Xin M, et al. Hippo pathway effector Yap promotes cardiac regeneration. Proc Natl Acad Sci USA. 2013; 110(34):13839-13844.
22. Xin M, et al. Regulation of insulin-like growth factor signaling by Yap governs cardiomyocyte proliferation and embryonic heart size. Sci Signal. 2011; 4(196):ra70.
23. Zhang X, et al. Yes-associated protein (YAP) binds to HIF-1α and sustains HIF-1α protein stability to promote hepatocellular carcinoma cell glycolysis under hypoxic stress. J Exp Clin Cancer Res. 2018; 37(1):216.
24. Zhu W, et al. Regenerative potential of neonatal porcine hearts. Circulation. 2018; 138(24):2809-2816.

What is claimed is:

1. A nanoparticle composition comprising:
(a) TT-10 or a pharmaceutically acceptable salt thereof and;
(b) nanoparticles comprising at least one polymer or copolymer.

2. The nanoparticle composition of claim 1, wherein the at least one polymer or copolymer is biocompatible.

3. The nanoparticle composition of claim 1, wherein the at least one polymer or copolymer comprises poly-lactic-co-glycolic acid (PLGA).

4. The nanoparticle composition of claim 3, wherein the PLGA has a molecular weight of from about 7000 to about 120,000 Da.

5. The nanoparticle composition of claim 3, wherein the PLGA has a ratio of lactide to glycolide of from about 3:1 to about 1:1.

6. The nanoparticle composition of claim 1, wherein the nanoparticles encapsulate the TT-10.

7. The nanoparticle composition of claim 1, wherein the nanoparticles have an average diameter of from about 75 nm to about 250 nm.

8. The nanoparticle composition of claim 1, wherein the nanoparticle composition comprises from about 2 to about 100 μM of TT-10 or the pharmaceutically acceptable salt thereof.

9. A method for improving at least one cardiac function or property following heart damage in a subject, the method comprising administering the nanoparticle composition of claim 1 to the subject.

10. The method of claim 9, wherein performing the method increases a level of at least one marker for cardiomyocyte cell proliferation in a cell or tissue relative to the level of the at least one marker prior to performing the method.

11. The method of claim 9, wherein the at least one marker comprises antigen KI-67 (Ki67), histone 3 phosphorylation (PH3), or any combination thereof.

12. The method of claim 9, wherein the subject is a human.

13. The method of claim 9, wherein administering the nanoparticle composition comprises intraperitoneal injection, oral administration, parenteral injection, intramuscular injection, intravenous administration, or any combination thereof.

14. The method of claim 9, wherein the nanoparticle composition is administered at a dosage of from about 0.5 to about 5 ng per kg of body weight of the subject.

15. The method of claim 9, wherein the at least one cardiac function or property comprises replacement of a myocardial scar with functional contractile tissue, cardiomyocyte renewal, cardiomyocyte cell-cycle activity, reduction in infarct size, reduction in infarct mass, a decline in cardiomyocyte apoptosis, angiogenesis, left ventricular ejection fraction (LVEF), left ventricular fractional shortening (LVFS), end-diastolic volume, end-systolic volume, stroke volume, or any combination thereof.

16. The method of claim 15, wherein infarct size comprises 15% or less of a ratio of an infarct area to a total left ventricular surface area, 4 weeks after performing the method.

17. The method of claim 15, wherein LVEF is at least 40%, 4 weeks after performing the method.

18. The method of claim 15, wherein LFVS is at least 20%, 4 weeks after performing the method.

19. The method of claim 9, wherein the heart damage is a result of myocardial infarction, infection, chemotherapy, heavy alcohol use, cardiac surgery, or any combination thereof.

20. The method of claim 9, wherein the nanoparticle composition is active at a target site for a period of at least 4 weeks.
